(19) United States
(12) Patent Application Publication
(10) Pub. No.: US 2022/0226260 A1
(43) Pub. Date: Jul. 21, 2022

(54) APPLICATION OF HONOKIOL AND MAGNOLOL IN PREPARATION OF MCR-1 ENZYME INHIBITOR

(71) Applicant: Jilin University, Changchun (CN)

(72) Inventors: XUMING DENG, CHANGCHUN (CN); YAN GUO, CHANGCHUN (CN); JIANFENG WANG, CHANGCHUN (CN); YONGLIN ZHOU, CHANGCHUN (CN); QIUSHUANG SHENG, CHANGCHUN (CN); NA LU, CHANGCHUN (CN)

(21) Appl. No.: 17/713,512

(22) Filed: Apr. 5, 2022

Related U.S. Application Data
(63) Continuation of application No. PCT/CN2020/106107, filed on Jul. 31, 2020.

(30) Foreign Application Priority Data
Oct. 6, 2019 (CN) 2019109465454

(51) Int. Cl. A61K 31/05; A61P 31/04

(52) U.S. Cl. CPC A61K 31/05; A61P 31/04

(57) ABSTRACT

An application of honokiol and magnolol in preparation of an MCR-1 enzyme inhibitor, provides a novel medical application of the honokiol and the magnolol in the preparation of the MCR-1 enzyme inhibitor. The honokiol and the magnolol can inhibit the activity of MCR-1 enzymes and restore the bactericidal activity of polymyxin to MCR-1 Enterobacteriaceae. In vivo experiments, the honokiol and the magnolol combined with polymyxin have a good therapeutic effect on bacterial infection expressing MCR-1, especially infections caused by MCR-1 positive Enterobacteriaceae, and have wide medical applications.

### TECHNICAL FIELD

The disclosure relates to the field of medical pharmaceutical technologies, and more particularly to an application/use of honokiol and magnolol in preparation of a mobilized colistin resistance (MCR-1) enzyme inhibitor.

### BACKGROUND

Increasing bacterial resistance has posed a serious threat to global public health, resulting in the emergence of many cases of bacterial infections that are difficult to treat, including those caused by carbapenemase-producing Gram-negative Enterobacteriaceae. With the emergence of super-resistant bacteria, treatment failures and high mortality rates against medicine-resistant bacteria, it has brought greater pressure to treat nosocomial infections, especially ICU infections. Polymyxin is the “last line of defense” for antibiotics therapy for medicine-resistant bacterial infections, but the discovery of MCR-1 has led to the unavailability of medicines for infections caused by polymyxin-resistant and carbapenem-resistant Enterobacteriaceae. At present, there are no reports of effective MCR-1 inhibitors. Therefore, it is of great significance to develop new safe and effective medicines for avoiding bacterial infections.

Honokiol and magnolol are mainly derived from the dry bark, root bark and branch bark of Magnolia officinalis Rehd. et Wils. or Magnolia officinalis Rehd. et Wils. var. biloba Rehd. etWils. of Magnoliaceae deciduous arbor plants, which has obvious and lasting pharmacological effects of relaxing central muscles, inhibiting central nervous system, resisting inflammation, resisting pathogenic microorganism, resisting ulcer, resisting oxidation, resisting tumor and the like. Besides the effects, honokiol also has the effects of resisting aging, Cholesterol lowering and other pharmacological effects, both of which play an important role in clinical medicine.

After searching, there is no relevant report on honokiol and magnolol in the preparation of MCR-1 inhibitors.

### SUMMARY

The disclosure provides a medical use of honokiol and magnolol in preparation of a mobilized colistin resistance (MCR-1) enzyme inhibitor, and discloses that honokiol and magnolol can inhibit the activity of MCR-1 enzyme and restore the bactericidal activity of polymyxin against MCR-1 positive Escherichia coli, Klebsiella pneumoniae and other Grain-negative bacteria.

Specifically, the honokiol of the disclosure has a molecular formula of C₁₈H₁₈O₂ and a molecular weight of 266.33. The magnolol has a molecular formula of C₁₈H₁₈O₂ and a molecular weight of 266.33.

The disclosure verifies that magnolol and honokiol can inhibit the activity of MCR-1 enzyme and restore the antibacterial activity of polymyxin against MCR-1 positive Enterobacteriaceae by chessboard minimum inhibitory concentration (MIC) test and time-kill curve method (also referred to as time-sterilizing curve). Furthermore, a mouse thigh muscle infection model is established to prove that honokiol and magnolol combined with polymyxin have a good therapeutic effect on infection caused by MCR-1 positive Enterobacteriaceae.

Compared with the related art, the embodiments of the disclosure may mainly have the following beneficial effects.

It provides a new medical use of honokiol and magnolol in the preparation of the MCR-1 enzyme inhibitor, and discloses that honokiol and magnolol can inhibit the activity of the MCR-1 enzyme and restore the bactericidal activity of polymyxin against MCR-1 Enterobacteriaceae. In vivo experiments, honokiol and magnolol combined with polymyxin have a good therapeutic effect on bacterial infection expressing MCR-1, especially infections caused by MCR-1 positive Enterobacteriaceae, and have wide medical applications.

### BRIEF DESCRIPTION OF DRAWINGS

FIG. 1 illustrates a time-kill curve of honokiol combined polymyxin against MCR-1 positive Escherichia coli of the disclosure.

FIG. 2 illustrates a time-kill curve of magnolol combined polymyxin against MCR-1 positive Escherichia coli of the disclosure.

FIG. 3 illustrates a colony colonization result of a mouse thigh muscle infection by honokiol combined with polymyxin of the disclosure.

FIG. 4 illustrates a colony colonization result of a mouse thigh muscle infection by magnolol combined with polymyxin of the disclosure.

### DETAILED DESCRIPTION OF EMBODIMENTS

The disclosure is further described by the following embodiments without limiting the disclosure in any way. Without departing from the technical solution of the disclosure, any alterations or changes made to the disclosure that are easy to be realized by those skilled in the art will fall within the scope of the claims of the disclosure.

### Embodiment 1

Honokiol and magnolol are used as mobilized colistin resistance (MCR-1) enzyme inhibitors in any pharmaceutically acceptable carrier.

### Embodiment 2

Honokiol and magnolol are used as MCR-1 enzyme inhibitors in preparation of medicines for treating infectious diseases.

### Embodiment 3

Honokiol and magnolol are used as MCR-1 enzyme inhibitors in treatment of bacterial infectious diseases, especially infections caused by MCR-1 positive Enterobacteriaceae.

### Test Example 1

### Minimum Inhibitory Concentration (MIC) Test

The antibacterial activities of honokiol, magnolol, polymyxin and their combination against MCR-1 producing Escherichia coli (also referred to as MCR-1 positive Escherichia coli) and Klebsiella pneumoniae are tested by chessboard method in 96-well sterile microplates, the MIC values are determined, and the fractional inhibitory concentration (FIC) indexes are calculated. Specifically, FIC=MIC (polymyxin combined)/MIC (polymyxin alone)+MIC (honokiol/magnolol combined)/MIC (honokiol/magnolol alone). The results are shown in Table 1 and Table 2:

TABLE 1
MIC and FIC values of honokiol combined with polymyxin for different strains

| | MIC (microgram per milliliter (μg/mL)) | | | | |
|---|---|---|---|---|---|
| | Polymyxin B | Polymyxin B | Honokiol | Honokiol | |
| Strains | alone | co-application | alone | co-application | FIC |
| MCR-1 positive | 8 | 1 | >512 | 32 | 0.1875 |
| Escherichia coli (E. | | | | | |
| coli DZ2-12R) | | | | | |
| MCR-1 positive | 32 | 2 | >512 | 32 | 0.125 |
| Klebsiella | | | | | |
| pneumoniae (K. | | | | | |
| pneumoniae ZJ02) | | | | | |
| MCR-1 engineering | 8 | 0.5 | >512 | 32 | 0.125 |
| bacteria E. coli | | | | | |
| BL21(DE3) | | | | | |
| (pET28a-mcr-1) | | | | | |
| E. coli BL21(DE3) | 1 | 0.5 | >512 | 32 | 0.5625 |
| (pET28a) | | | | | |

TABLE 2
MIC and FIC values of magnolol combined with polymyxin for different strains

| | MIC (μg/mL) | | | | |
|---|---|---|---|---|---|
| | Polymyxin B | Polymyxin B | Magnolol | Magnolol | |
| Strains | alone | co-application | alone | co-application | FIC |
| MCR-1 positive | 8 | 2 | >512 | 32 | 0.3125 |
| Escherichia coli (E. | | | | | |
| coli DZ2-12R) | | | | | |
| MCR-1 positive | 32 | 8 | >512 | 32 | 0.3125 |
| Klebsiella | | | | | |
| pneumoniae (K. | | | | | |
| pneumoniae ZJ02) | | | | | |
| MCR-1 engineering | 8 | 1 | >512 | 32 | 0.1875 |
| bacteria E. coli | | | | | |
| BL21(DE3) | | | | | |
| (pET28a-mcr-1) | | | | | |
| E. coli BL21(DE3) | 1 | 1 | >512 | 32 | 1.0625 |
| (pET28a) | | | | | |

In summary, honokiol or magnolol alone have no antibacterial effect, but the combination of honokiol or magnolol with polymyxin can reduce the MIC value of polymyxin to the engineering strain E. coli BL21 (DE3) (pET28a -mcr-1) expressing MCR-1, and has an obvious synergistic effect. However, in the engineering strain E. coli BL21 (DE3) (pET28a) that does not express MCR-1, the combination of honokiol or magnolol with polymyxin does not have any synergistic effect, and indicating that honokiol or magnolol is an effective MCR-1 inhibitor. Consistent with the above results, the combination of honokiol and polymyxin can reduce the MIC value of polymyxin by 8-fold for MCR-1 positive Escherichia coli and 16-fold for MCR-1 positive Klebsiella pneumoniae. The combination of magnolol and polymyxin can reduce the MIC value of polymyxin by 4-fold for MCR-1 positive Escherichia coli and 4-fold for MCR-1 positive Klebsiella pneumoniae. FIC values indicate that honokiol or magnolol is synergistic with polymyxin in MCR-1 positive strains.

### Test Example 2

### Time-Kill Curve (Also Referred to as Time-Sterilizing Curve) Test

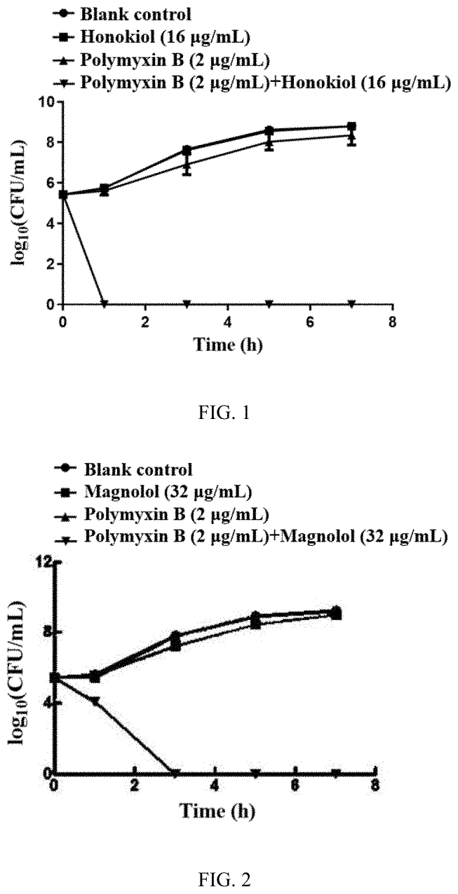

An overnight culture of MCR-1 positive Escherichia coli isolate is taken and adjusted to 1×10⁷ colony forming units per milliliter (CFUs/mL) for use. Sterile test tubes are taken and divided into 4 groups (including blank control group, 2 μg/mL polymyxin group B group, 16 μg/mL honokiol or 32 μg/mL magnolol group, and 16 μg/mL honokiol or 32 μg/mL magnolol combined with 2 μg/mL polymyxin B group), each group is marked for 1, 3, 5 and 7 hours. 1 mL of autoclaved Lysogeny broth (LB) medium is added into each of the test tubes, and then 10 microliters (μL) of adjusted bacterial solution are added into each of the test tubes so that the concentration of bacterial solution in each of the test tubes is 1×10⁵ CFUs/mL. Among them, 2 μg/mL polymyxin group B group, 16 μg/mL honokiol or 32 μg/mL magnolol group, and 16 μg/mL honokiol or 32 μg/mL magnolol combined with 2 μg/mL polymyxin B group are added with the corresponding amount of antibiotics and inhibitors respectively. Immediately after mixing, the bacterial solution of the antibiotic-free control group is coated and counted as the CFU at 0 H. After that, the bacterial solution in the corresponding test tube is taken every 1, 3, 5 and 7 hours, and the plate is coated for counting, and the time-kill curves are drawn, as shown in FIG. 1 and FIG. 2.

In summary, honokiol combined with polymyxin can completely kill MCR-1 positive Escherichia coli within 1 hour, and magnolol combined with polymyxin can completely kill MCR-1 positive Escherichia coli within 3 hours.

### Test Example 3

### Colony Colonization Experiment of Mouse Thigh Muscle Infection

### Mouse Model of Klebsiella pneumoniae Thigh Muscle Infection

After BALB/C mice (female, about 20 grains (g)) are restrained, MCR-1 positive Klebsiella pneumoniae suspension (2×10⁷ CFUs) is injected through the inner thigh to establish a mouse thigh muscle infection model.

### Colony Colonization Experiment

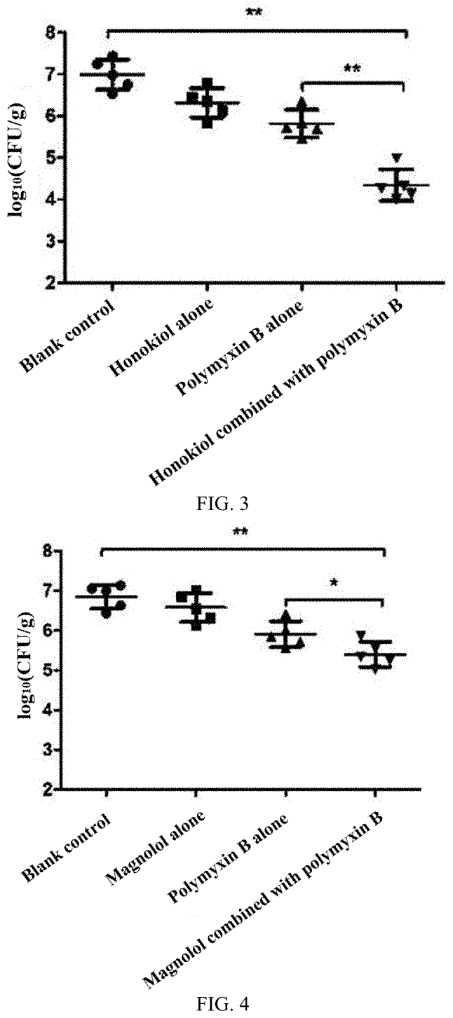

After intramuscular injection of MCR-1 positive Klebsiella pneumoniae into the inner thigh of mice, 5 micrograms per kilogram (mg/kg) (50 μL) of polymyxin B dissolved in sterile ultra-pure water (dd H₂O), 50 mg/kg (50 μL) honokiol or magnolol dissolved in dimethyl sulfoxide (DMSO) solution, and 50 mg/kg of honokiol or magnolol combined with polymyxin B (5 mg/kg) are injected subcutaneously, while the positive control group is subcutaneously injected with 50 μL DMSO blank solvent. All groups of mice are administered subcutaneously every 8 hours for three times. After 36 hours of administration, the mice are sacrificed, and the thigh muscles are weighed, homogenized, diluted and counted. The results are shown in FIG. 3 and FIG. 4.

In summary, after treatment of honokiol or magnolol combined with polymyxin B, the number of bacterial colonization in mouse thigh muscle is significantly reduced, and there is no significant effect with magnolol, honokiol, or polymyxin B alone.

What is claimed is:

1. A use of honokiol and magnolol, wherein the honokiol and the magnolol are used to prepare a mobilized colistin resistance (MCR-1) enzyme inhibitor.

2. A use of honokiol and magnolol as active ingredients, wherein the honokiol and the magnolol as the active ingredients are used to prepare a medicine for treating infectious diseases.

3. The use of the honokiol and the magnolol as the active ingredients in the preparation of the medicine for treating the infectious diseases according to claim 2, wherein the infectious diseases are diseases caused by bacterial infection.

4. The use of the honokiol and the magnolol as the active ingredients in the preparation of the medicine for treating the infectious diseases according to claim 3, wherein the bacterial infection is an infection caused by Grain-negative Enterobacteriaceae carrying MCR-1 on a plasmid.

5. An MCR-1 enzyme inhibitor prepared with honokiol and magnolol as active components, wherein the honokiol is a pharmaceutically acceptable carrier.
